(19) United States
(12) Patent Application Publication
(10) Pub. No.: US 2019/0311790 A1
(43) Pub. Date: Oct. 10, 2019

(54) Patient Care System

(71) Applicant: CaregiversDirect, a Public Benefit Corporation, Los Angeles, CA (US)

(72) Inventors: Neama Dadkhahnikoo, Los Angeles, CA (US); Joshua Greer, Beverly Hills, CA (US)

(21) Appl. No.: 16/377,011

(22) Filed: Apr. 5, 2019

Related U.S. Application Data
(60) Provisional application No. 62/654,293, filed on Apr. 6, 2018.

(51) Int. Cl. G16H 10/60; G16H 50/20; G16H 80/00

(52) U.S. Cl. CPC G16H 10/60; G16H 15/00; G16H 80/00; G16H 50/20

(57) ABSTRACT

A caregiving system and method used in caring for a patient is provided. The design includes receiving information regarding the patient, recording the information received, and based on the information received, determining a query applicable to the patient, wherein the query includes actionable data with respect to the patient. Determining the query applicable to the patient includes employing artificial intelligence to determine possible care actions based on attributes of the patient and available care solutions, employing artificial intelligence training based on prior received applicable data to improve potential query related outcomes, and establishing the query based on query selection data, datapoint value metrics, and response rate data.

The present application claims priority based on U.S. Provisional Patent Application Ser. No. 62/654,293, filed Apr. 6, 2018, inventors Neama Dadkhahnikoo, et al., entitled “Patient Care System,” the entirety of which is incorporated herein by reference.

### BACKGROUND OF THE INVENTION

### Field of the Invention

The present invention generally relates to the art of data capture systems, and more particularly to systems and devices used to facilitate, diagnose and predict patient care.

### Description of the Related Art

A number of medical systems and devices are available that facilitate use of medical patient records and record keeping. One major problem with these systems is the inability to interact with other systems. Other issues include the proprietary nature of such records and the risks associated with record keeping, including issues of patient confidentiality, identity theft, and failure to provide accurate representations of the issues involved. As an example, a patient may prefer one caregiver to another, or may get better results from one medication than another, and such information may not be included in his or her record. There is typically no guarantee of, or ability to provide, continuity of care in existing solutions.

One underserved area is caregiving to patients in their homes. Existing caregiving systems are typically hospital based and rarely apply in home care situations. Some caregiver entities maintain a platform that captures patient information during homecare visits, e.g. pulse, temperature, blood pressure, medications taken, and so forth. Caregiving entities have, for example, employed a notebook that remains in the home of each client. Caregivers physically write client information in the notebook, and that information is helpful when caregivers change shifts, or different caregivers are sent to the home, etc. These notes are often not cycled back to the agency for any purpose, such as review or analysis.

Online caregiving entities, and certain caregivers without an online presence, employ a basic device application that schedules and provides a checklist for caregivers' daily tasks. Caregivers can log wellness information into the application. These types of applications typically employ a standardized list of questions that are answered using the application, i.e. “how was Ms. Smith's mood today?” These types of simplified applications tend to be limited in scope.

The previous ways of collecting information about patients serviced by caregivers, such as in-home caregivers, are limited and have been stagnant for several years. Such systems do not maximize user participation and offer limited accuracy and use for much of the data obtained. Several potential inputs are outright ignored, e.g. duration of time since each query data point has been collected, response rate, the value of each query data point in predicting, and inputs and queries from trusted sources and physicians.

Thus, there is a need to provide a system that overcomes the drawbacks identified above.

### SUMMARY OF THE INVENTION

Thus according to the present design, there is provided a caregiving system. The system includes a user device, a server arrangement connected to the user device, a query recording system connected to the server arrangement configured to record caregiver queries and responses to caregiver queries and provided recorded results to a storage element, and intelligence preparation hardware configured to collect data from the storage element and form caregiver queries based on query selection data, data point value metrics, and response rate information using at least one training system. Queries formed by the intelligence preparation hardware are provided to the server arrangement and user device to solicit query responses.

According to a further aspect of the present design, there is provided a patient caregiving system comprising a server arrangement configured to receive information and data from at least one user device, a data storage element comprising a query recording system configured to receive a care query from the server arrangement and store the care query, and a query processing module configured to receive query data from the data storage element and provide selected queries to the server arrangement, wherein the query processing module comprises a query selection system configured to employ query selection data, datapoint value metrics, and response rate data to select a query and provide the query to the server arrangement for transmission to a receiving user device.

According to another aspect of the present design, there is provided a method for providing caregiving information used in caring for a patient, comprising receiving information regarding the patient, recording the information received, and based on the information received, determining a query applicable to the patient, wherein the query comprises actionable data with respect to the patient. Determining the query applicable to the patient comprises employing artificial intelligence to determine possible care actions based on attributes of the patient and available care solutions, employing artificial intelligence training based on prior received applicable data to improve potential query related outcomes, and establishing the query based on query selection data, datapoint value metrics, and response rate data.

Various aspects and features of the disclosure are described in further detail below.

### BRIEF DESCRIPTION OF THE DRAWINGS

FIG. 1 is a general overview of the present system;

FIG. 2 illustrates the general functionality of the user interface;

FIG. 3 represents data storage functionality;

FIG. 4 is data preparation (AI data preparation) functionality;

FIG. 5 shows a functional representation of the AI database;

FIG. 6 is a functional representation of the data value algorithm; and

FIG. 7 is a functional overview of the query algorithm.

### DETAILED DESCRIPTION

The following detailed description is of the best presently contemplated modes of carrying out the invention. This description is not to be taken in a limiting sense, but is made merely for the purpose of illustrating general principles of embodiments of the invention. The scope of the invention is best defined by the appended claims. In certain instances, detailed descriptions of well-known devices and mechanisms are omitted so as to not obscure the description of the present invention with unnecessary detail.

The present design is a system that is configured to capture significant information such as the physical, mental, social, and behavioral information generated (in time-series) during facility care, such as home care, by care recipients and care providers. The system is configured to transform the data received into actionable data.

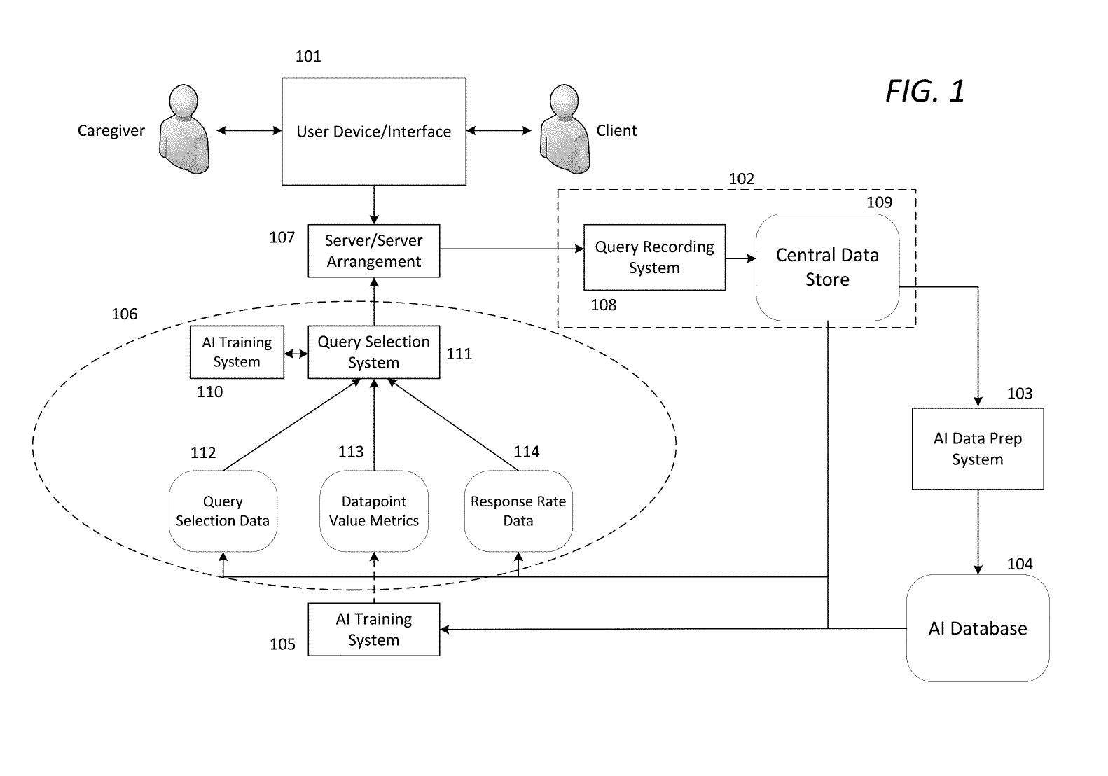

Generally, the system consists of six subsystems shown in FIG. 1, including a user interface 101 on a device, including but not limited to a smartphone or computing device, that displays queries to clients and caregivers, together or separately, and records the responses. Other devices may be employed, such as home assistant computing devices, health devices, smart watches, and the like. Information from user interface 101 may be transmitted to a server, that then interfaces with data storage 102 that may include a query recording system 108 and a central data storage element 109. Data storage 102 captures the per shift query information and other client and caregiver data points and stores this information to a central data store element. Preparation module 103, which handles artificial intelligence preparation, transforms data from the central data storage element 109 into both sparse and dense data formats suitable for use by artificial intelligence (AI) processes. The system also includes an AI database module 104, or AI storage module, that stores sparse and dense data formats capturing the physical, mental, social, and behavioral data points of clients and their caregivers in time series, evaluates, processes, and links these client records to other records by cohort and family. Also provided is a deep learning neural network module 105 that receives and processes client data and possible client outcomes to predict the importance of the different data categories queried. The system further includes a query module 106 that decides those queries that may or will appear to a client and caregiver on a certain basis, such as on a per shift basis, using a number of local and global variables. Query module 106 includes AI training system 110, query selection system 111, query selection data 112, data point value metrics 113, and response rate data 114.

As may be appreciated from FIG. 1, data is retrieved from user devices at user interface 101 and provided to server 107, which routes the information to data storage 102, preparation module 103, AI database module 104, deep learning neural network module 105, also known as an AI training system, and Query module 106, wherein the results of query selections are provided back to server 107 and potentially to the user devices in user interface 101. In this manner, information is received, assessed, current conditions and situations may be analyzed and determined, and information provided to caregivers and other interested individuals as warranted under the circumstances.

Older methods of patient narrative tracking have been stagnant, failing to employ interactive functionality, such as interactive queries, to maximize user participation and accuracy of data. The downside is easily seen: keeping notebooks at the location of the patient restricts the knowledge available about the patient and is potentially detrimental to the patient, and dynamic ability to deploy care based on circumstances is virtually nonexistent. There are also several potential inputs that are ignored, including but not limited to data such as duration of time since each query data point has been collected, response rate, and the value of each query data point in predicting insights regarding patient care.

The purpose of the system is to dynamically collect and employ an ongoing record of the status and progress (or regress) of the client receiving care, the care provider, and the care service being provided. The system receives, transforms, and store this data as actionable data. Such data is then available for use by artificial intelligence and machine learning processing.

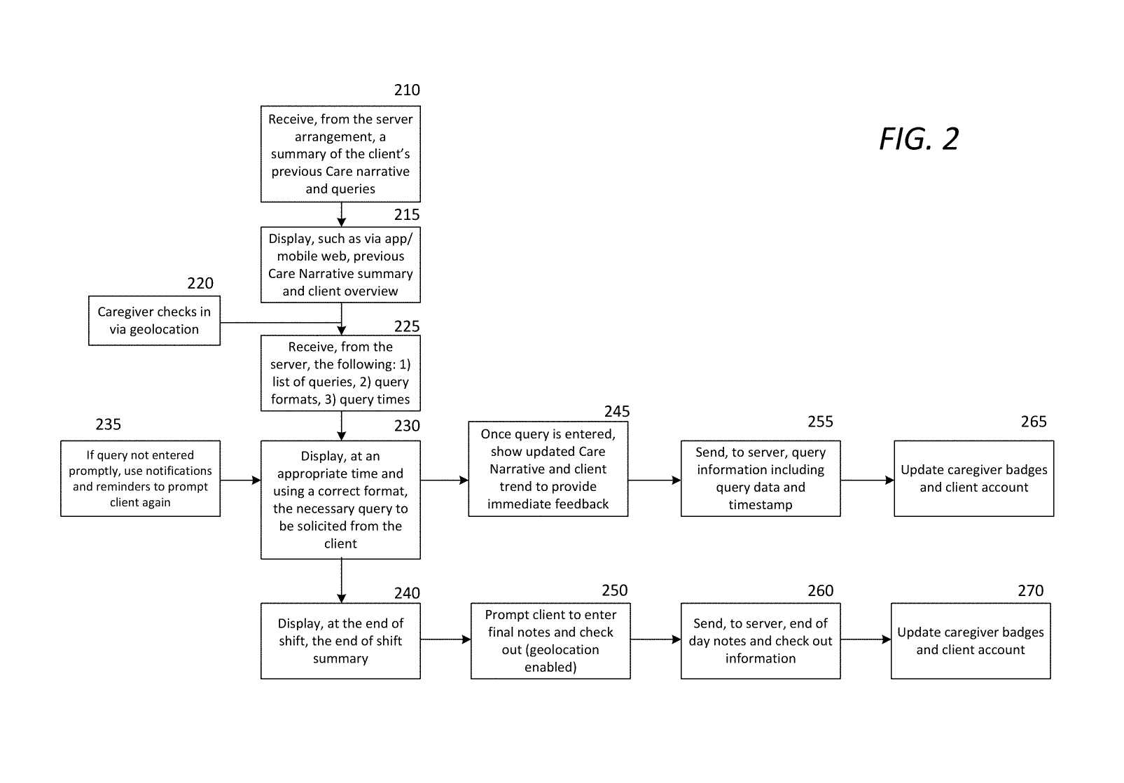

FIG. 2 illustrates a version of the graphical user interface functions performed by user interface 101. According to FIG. 2, the user interface system or arrangement shown is the main point of interaction between the caregiver providing care for the client and the other components of the system. The user interface 101 can be accessed on a device, including but not limited to a smartphone or computing device, through a web browser or app. Before a caregiver's shift, the user interface 101 may use the server 107 and its application programming interface to retrieve at point 210, a summary of a patient's or client's care narrative. The user interface 101 then uses the mobile app or web browser display at point 215 to show the information to the caregiver. The displayed information allows the caregiver to obtain a quick impression or snapshot of the client's health and psychosocial information. Such information allows the caregiver to better prepare for treating the patient or client, such as in preparation for an upcoming shift.

The caregiver may use geolocation at point 220 to check in, or establish presence at a desired location, through the mobile app or web browser. At point 225, the user interface 101 uses the server 107, such as its application programming interface, to retrieve the care shift's necessary queries. Queries in this instance may include, for example, the physical, mental, social, and behavioral information regarding a patient or client. As may be appreciated, varying levels and types of queries may be available, and they may be grouped or categorized. Information provided may include how the queries are formatted when displayed, and the time when the query should be displayed, if applicable. When the assigned time is reached, shown by point 230, the user interface 101 may display the query, in the proper format, to the caregiver. Queries may take many and various forms. Some examples of some queries and their formats include: “What recreational activities did [first name] do today? Check-list: Cooking, Artwork, Crafts, Music, None, Other (specify)” “In which areas did [first name] exhibit improvement today? Check-boxes: Memory, Mobility, Communication, Mood, Pain, Other (explain), None” “With whom did [first name] interact with today, besides you? Check-list: Family member, Friend or neighbor, Health professional, Clergy, Other (explain).”

The user interface 101 may display multiple types of interactive query types and query designs, such as vital statistics, weight, blood pressure, pulse, and so forth. The user interface 101 may include space for free form annotations. Query variety may be employed as static and/or predictable queries often result in end-user fatigue, which in turn reduces response rates and leads to inaccurate responses. Thus varying user interface designs, query formatting, and the use of gamification can provide a successful data gathering system. If the caregiver does not respond to the query within an assigned or predetermined time, at point 235 the notification system may be activated and use mobile notifications and reminders, including but not limited to text messages, to remind the caregiver to complete the query. Once the caregiver completes the query with the appropriate information, the user interface 101 may use the mobile device or computing apparatus to display the updated Care Narrative and associated client trend data to the caregiver, shown by point 245. Displaying this information after submitting the query can provide positive and immediate feedback to the caregiver's actions.

Additionally, the user interface 101 may use the application programming interface of server 107 to submit, the completed query information data and appropriate timestamp to the Data Storage system described below, shown by block 155. Server 107 may be a single server or server arrangement, and the terms are used interchangeably herein and may represent in any scenario one or more servers. The user interface 101 may update the mobile app or web browser display at point 265 with updated caregiver badges, client badges, and client information as desired. Once an end of a care shift is reached, the user interface 101 uses the mobile app or web browser display at point 240 to display the end of shift summary for the caregiver. The end of shift summary summarizes the day's care shift, activities, queries, and may provide other relevant information. The user interface 101 then uses the mobile app or web browser display at point 250 to prompt the caregiver to provide end of shift notes and to check out of the care shift. The check out process is geolocation enabled to allow for tracking and auditing of care shift completion for the benefit of the caregiver and the client. Once the caregiver submits the end of day care notes for the Care Narrative, the user interface 101 may employ the application programming interface of server 107 to submit, at point 260, the completed end of day notes and other check out information to the data storage element 102, described in more detail below. The user interface 101 may update the mobile app or web browser display at 170 with updated caregiver badges, client badges, and client information as necessary. Badges in this context include information relevant to the completed shift and pertinent to the individual (caregiver or patient)—such as time spent, procedures covered, time in, time out, etc.

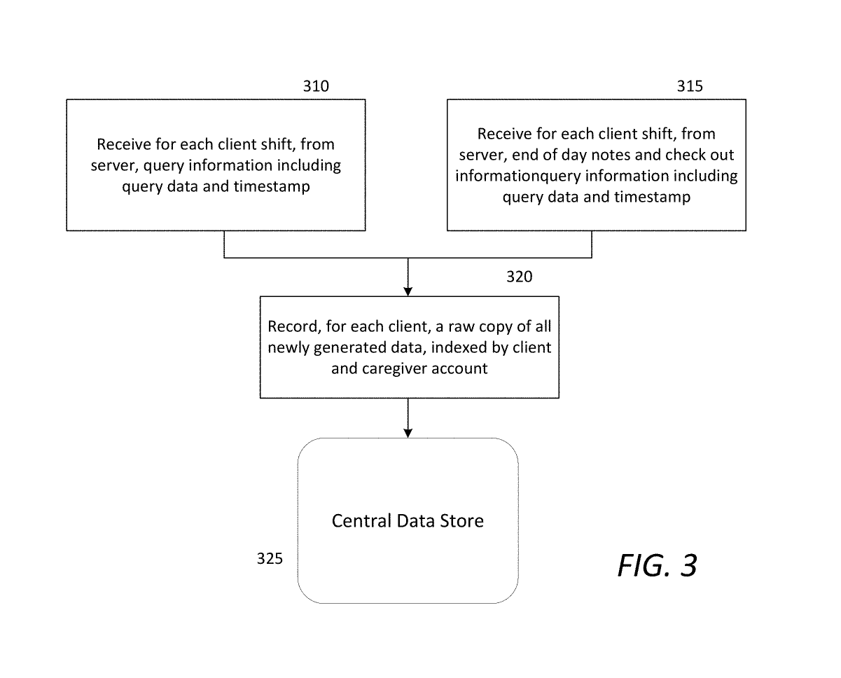

The data storage system 102, shown in detail in FIG. 3, receives information from the user interface 101 through server 107 and stores the information in central data store 109. For each care shift provided by the caregiver to the client, the caregiver and possibly the client complete and submit multiple queries. At point 310, data storage system 102 receives each submission of query information data and associated timestamp at box 310, through the application programming interface of server 107. Database management system at point 320 then records client and caregiver records to central data store 325. In addition, for each care shift, the caregiver provides end of shift notes and checkout information. The Data Storage data storage system 102 receives at box 315, through server 107's application programming interface, each submission of end of shift notes and checkout information. Database management system 320 records this information to the central data store 325 for the proper client and/or caregiver.

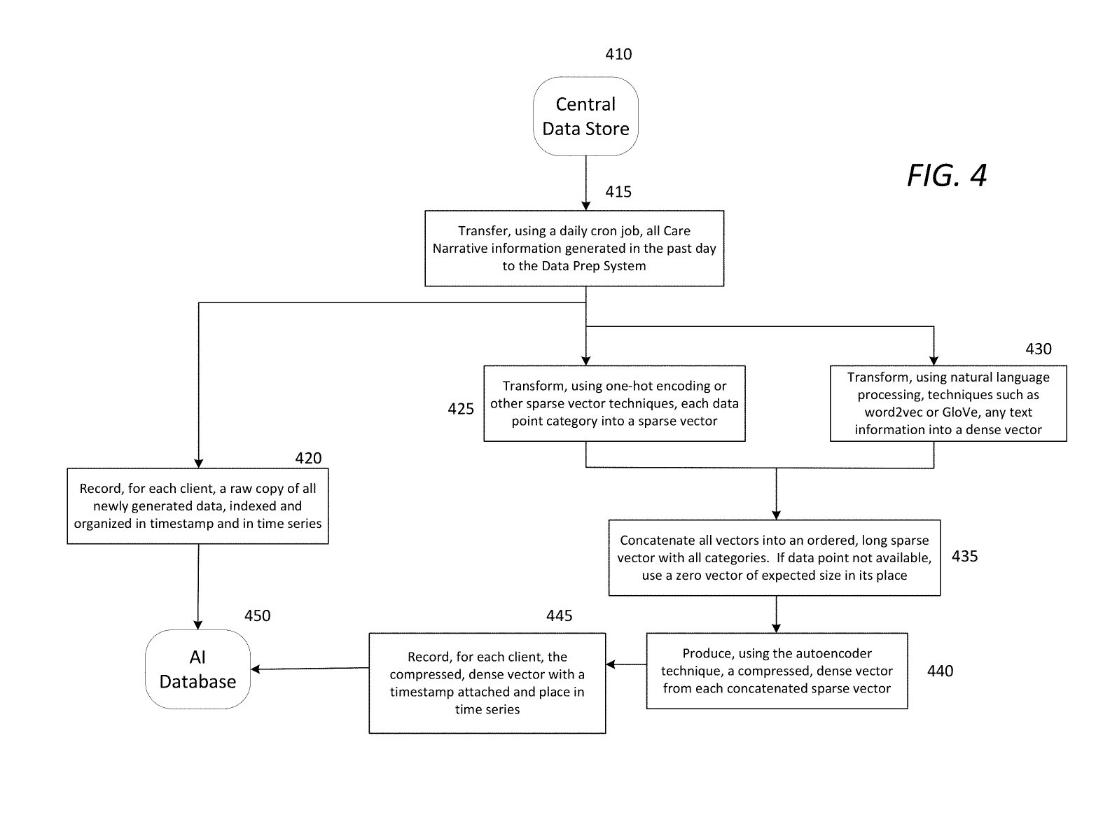

FIG. 4 shows the AI data prep system 103. AI data prep system 103 takes the newly data generated within a specified time period (e.g. daily) from the central data store 109, transforms the data, and then records the data to an AI database. The system uses the database transfer process at point 415 to transfer all care narrative data generated within the set time period from the central data store 109 to the AI data prep system 103 for processing, shown as central data store 410 in this view. The transfer takes place on a regular basis through a cron job or similar scheduled transfer process. As known to those skilled in the art, a cron is a time-based task scheduler, typically in Unix.

Once the data is imported, the system initiates two parallel data processes. First, at point 420, the system records the newly generated data, and provides the data to the AI database 104, also shown as AI database 450, in a raw (original), unprocessed format. The system adds this new data to the client record and places the data in the correct index position based on the timestamp attached to the data. The result is a time series record of all raw physical, mental, social, and behavioral data generated by the client's home care up until the current date, stored in the AI database 104 for future use. Second, the system uses two encoding processes to transform the imported data into vector space, a mathematical representation of information through the use of vectors, that allow for the vector mathematics in establishing and assessing related information, such as shift assignments, patient care tasks, patient questions, and so forth. Natural language encoding process 425 transforms the data point(s) for each data category into an appropriately encoded sparse vector, through the use of one-hot encoding or other similar sparse vector encoding techniques. At the same time, encoding process 430 transforms text found in the data (for example, the end of day notes) into a dense vector representation, by use of dense word vector encoding techniques such as word2vec or GloVe. These dense word vector encoding models are in certain circumstances pre-trained with outside data or trained using internally generated data, or a combination of both. Once the system has completed both encoding processes for a single client record, vector transformation process element 435 concatenates all of the sparse and dense vectors generated into a single ordered, long sparse vector. The transformation process ensures that the order of the data appearing in the concatenated vector is the same. The system replaces any missing data or empty vector with an empty vector of the same size as the vector that would be generated for that category. The end result is a very long, mostly sparse vector that contains all the information generated for a single client since the last cron job time period (e.g. 24 hours).

Vector transformation process 440 takes as input the ordered long, sparse vector and transforms the vector into a dense vector through the use of an autoencoder. The autoencoder learns a representation (encoding) for a set of data, typically for the purpose of dimensionality reduction. Each client's long, sparse vector is thus transformed into a dense vector that is more useful for certain AI processes. A database management system 445 then attaches the appropriate timestamp to the dense vector and, for each client, places the vector in the AI Database 450. The vector, which represents the new data generated for the client, is added to the client record and placed in the correct index position based on the timestamp attached to the vector. The result is a time series of dense vector-space representations of all physical, mental, social, and behavioral data generated by the client's home care up until the current date, stored in the AI Database for future use.

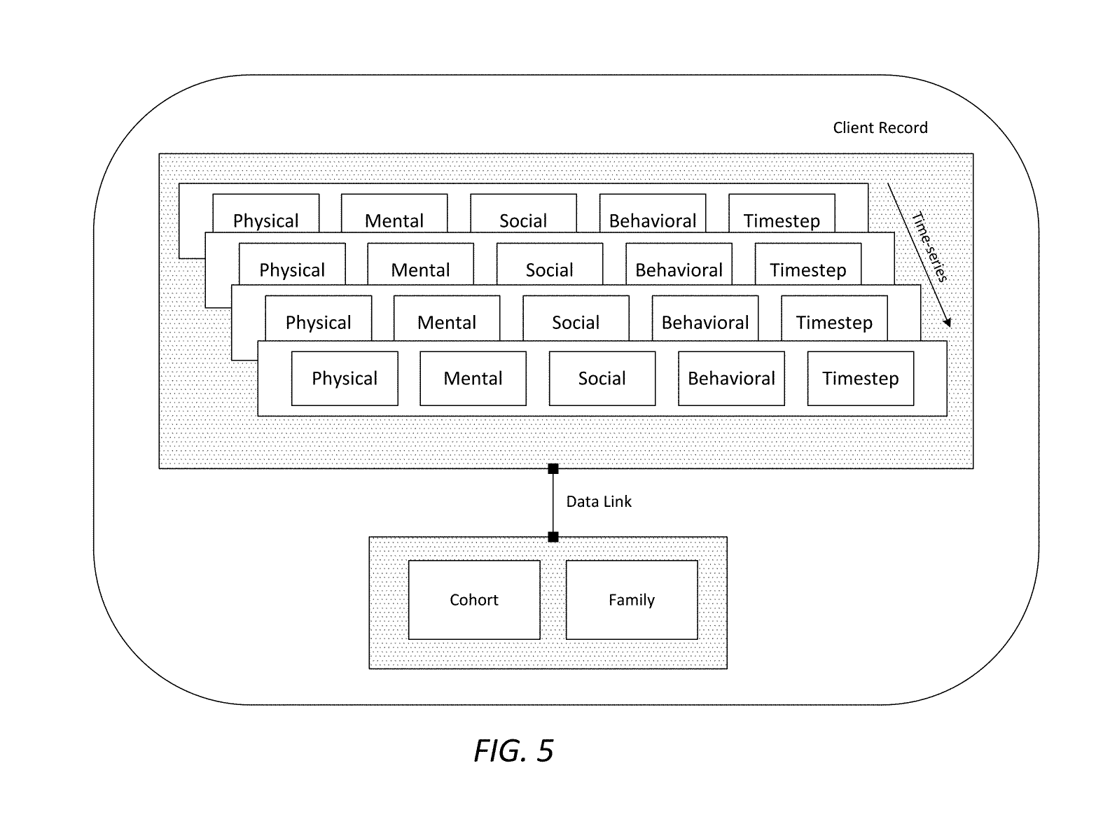

FIG. 5 shows a representation of the AI database. AI database 104 or AI database 450 contains a record for each client of his or her physical, mental, social, and behavioral data generated during the home care of the client or patient by a caregiver. Each client record includes a time series of all the information gathered over time, providing a rich health record of data that can be used for the purpose of improving the care of clients, improving health outcomes, reducing readmissions, and predicting the onset and progression of disease. Data can be fixed or requested from other sources, including but not limited to physicians, hospitals, and the like. The data is stored in AI database 104 or 450 as raw, original data and as vector-space representations of the data. All data is associated with a timestamp, incremental timestep, or other time sorting mechanisms. Additionally, each client record is associated with a cohort (a group of clients that share common characteristics or experiences within a defined time-span) and a family (clients that are family members).

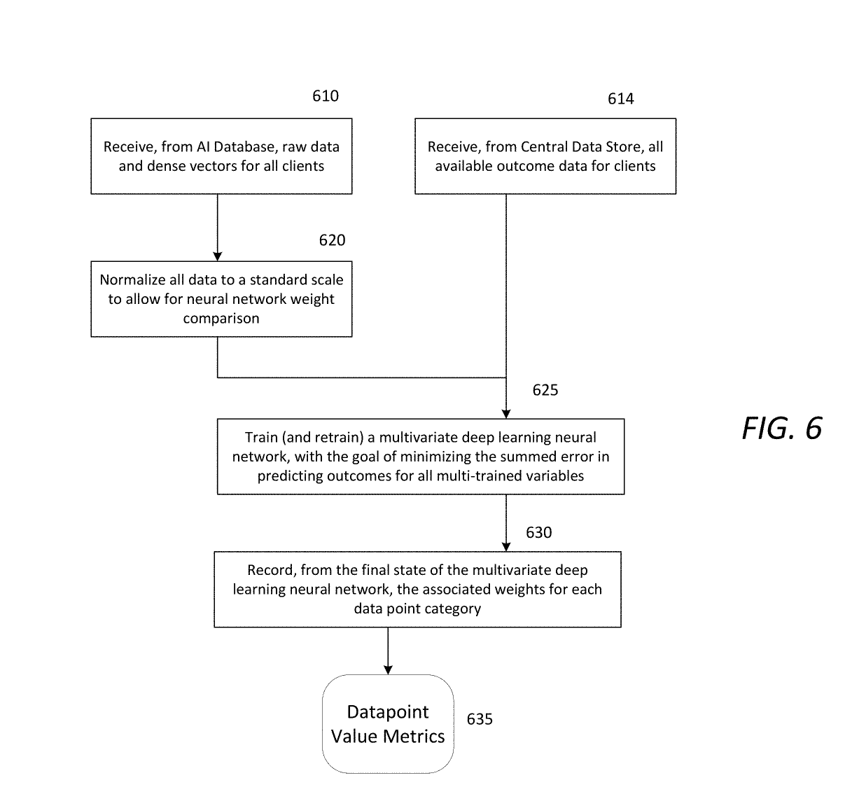

The data value algorithm system, or AI training system 105, is shown in detail in FIG. 6. The data value algorithm system uses a deep learning neural network to determine which data points are most valuable in predicting specific target outcomes for clients. At point 610, the AI database, such as AI database 450, transmits the raw data and vector representations of all clients to the data value algorithm. The normalization process 620 then normalizes the data by mapping all numeric values to a similar number scale (e.g. 0 to 1), allowing for later cross-category comparisons. At point 615, the AI database 450 transmits all available outcome data to the data value algorithm. The type of outcomes collected by the system may be determined based on client, caregiver, or business needs (for example the system may collect outcome info on client readmission after discharge from a hospital, client disease diagnosis, or caregiver termination). The system uses the normalized client data and client outcomes to train and retrain a neural network 625. The neural network 625 may be a multivariate deep learning neural network trained on all client home care data with a goal of minimizing the summed error of predicting specific outcomes of clients. Other types of neural networks, such as a convolutional neural network or a long short-term memory network, can also be used. Once the system has trained the neural network, the data recording process 630 captures the associated weights for each data category from the final state of the trained neural network and records these associated weights to the data point value metrics database as shown at point 635. The system, including the neural network 625, may be used to match caregivers with patients.

With the data already normalized, the associated weights of the neural network can be used to provide an estimate of the importance of each category and determine the outcome desired. As an example, with scheduling of caregivers, weighting can be provided to a caregiver who has been working with a particular patient or client. If the caregiver and patient have a long term, high quality relationship, a high weighting value can be provided, while no relationship or a one time encounter or a relationship flagged as being unacceptable can be accorded a low weighting. Availability of the caregiver can be weighted and considered, as well as preferences. For example, if patient X has a preference for female caregivers within a five mile radius of her home, this can be weighted higher than one who is 20 miles from her home. The weightings, availability, and other relevant factors can be evaluated to determine a best candidate for a particular care situation. At point 635, the system can import data point value metrics from hand-crafted values derived from subject matter experts or other external data sources.

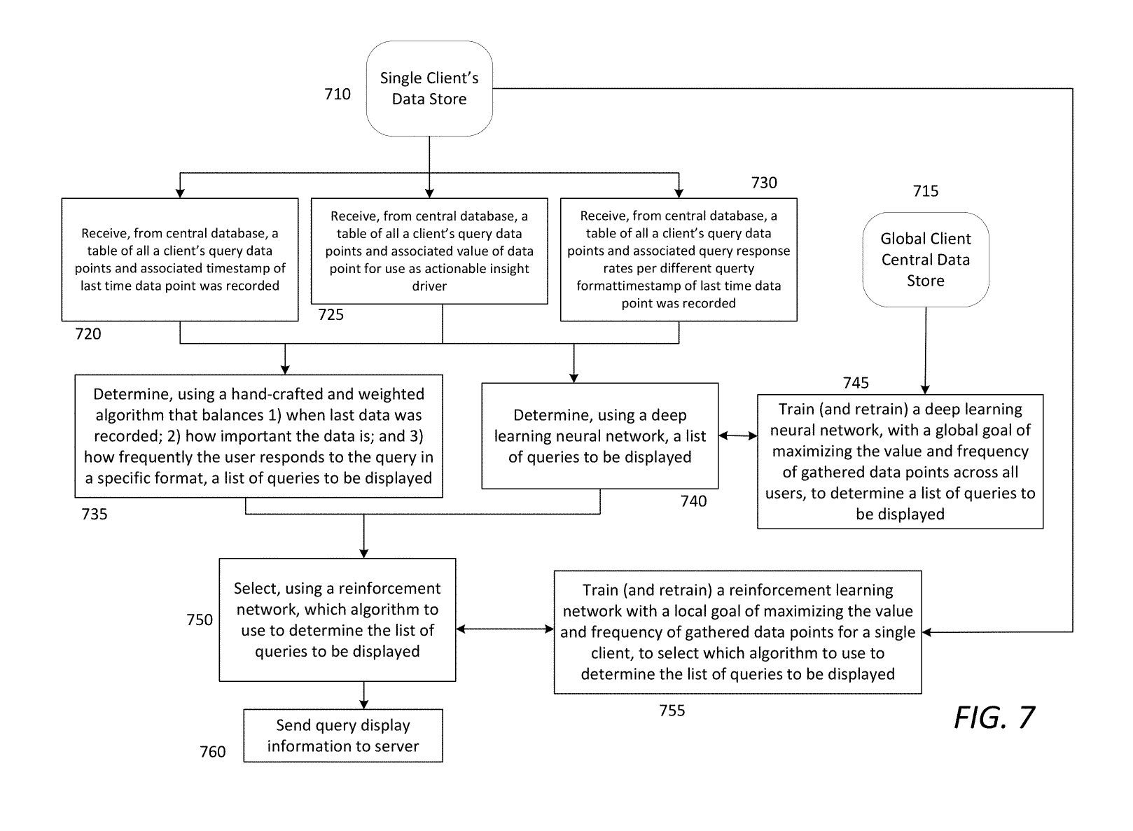

The query algorithm system, shown in FIG. 7, employs weighted algorithms and neural networks trained on global data, in conjunction with a policy network trained on local data, and determines the query formats that may be displayed for each client's care shift in order to maximize the value of the data being gathered. The query algorithm system uses two parallel query determination algorithms First, for each client's upcoming home care shift, the system accesses the client's home care record 710 from the AI database. Using this client record, the system receives multiple items. At point 720, the system receives a table or record that documents the last time each data category was last accessed (e.g. 46 hours for “Client's measure of social interaction with friends”). At point 725, the data value algorithm determines a table of the data value for each query category, while at point 730, the system determines a table of the caregiver or client's response rate for a data category when associated with a query format. For example, a caregiver who fails to ask a particular question repeatedly is noted. The question may be poorly worded, or the caregiver may be specifically instructed to ask the question together with a discussion of the importance of the question. The system then passes this data to the weighted algorithm at point 735. The weighted algorithm balances when data was last recorded, relative importance of the data, and how frequently the caregiver responds to the query when formatted in a specific way. In this manner, the weighted algorithm determines the queries and formats displayed for a subsequent shift. The weighted algorithm can be created manually, and/or can be designed using statistical methods such as regression. The weighted algorithm may be periodically modified to improve performance In the parallel query determination algorithm process, the system retrieves all global client data from the AI database at point 715 from the AI database and uses the data to train and retrain a neural network at point 745. One example of such a network is a deep learning neural network trained on all client home care and query data with a goal of maximizing total data value when selecting query categories and query formats. Such a network, for example, uses a differentiable formula that calculates the value of each home care shift based on the data value from the data value algorithm, the actual response rate, and the data collection frequency, and then trains to maximize this value across all client shifts and their underlying query lists and query formats. Other types of formulas to maximize the value of the data gathering process and other types of neural networks, such as a convolutional neural network or a long short-term memory network, can be employed.

The resulting trained neural network shown at point 740 uses the following as input: a table that documents when the last time each data category was last accessed, shown as point 720; a table of the data value for each query category as determined by the Data Value Algorithm, shown as point 725; and a table of the caregiver or client's response rate for a data category when associated with a query format, shown at point 715. The system then determines queries and formats to be displayed for a subsequent shift. The system submits the results of the parallel query determination algorithms to the reinforcement learning network at point 750. The reinforcement learning network may select a preferred algorithm for each client's home care shift. Reinforcement learning in this instance may be a machine learning technique that uses an agent to act to maximize rewards. The Query Algorithm trains and retrains a reinforcement learning network 755 to maximize the value of the data gathered for each individual client based on his or her previous home care record 710. This reinforcement learning network then weighs the suggestions of each of the parallel query determination algorithms in the context of the specific client and his or her upcoming home care shift, and may select a preferred algorithm. The selected algorithm's output of preferred queries and query formats is then transferred via server 107 to the user interface 101.

Thus FIG. 1 shows a general system for collecting, recording, and processing records related to caregiving, wherein the caregiver and/or client/patient can employ a user device to receive queries and respond to queries, shown as user interface 101. Server 107 interfaces the user interface 101 with back end processing, which includes data storage 102. Data storage 102 provides data to AI data prep system 103 and query algorithm 106. AI data prep system 103 acquires and prepares data for processing, such as relevant caregiver and/or patient entries used in efficiently assessing workload and personnel and deploying caregivers on an as needed basis, including schedules, abilities, and so forth. In one instance, AI data prep system 103 may retrieve the schedules for all relevant personnel, current issues with particular clients/patients, wherein those issues are graded (e.g. critical, non-critical, minor, etc.), and may also retrieve relevant answers provided to recent queries or past queries. AI related information is stored in AI database 104, which as differentiated from data storage 102 is a subset or alternate set of data related to AI processing. Again, one example of data provided on AI database 104 may be patient care shift needs, e.g. patient X needs a caregiver from either 9-noon on Tuesday or 3-5 on Thursday. This information may also be maintained on data storage 102, but is readily available at AI database 104 for processing by the AI portion of the design.

AI training system 105 then trains the data, such as evaluating caregiving history (schedule, personal preferences, and so forth) to provide best information for subsequent processing. Query algorithm 106 includes a query selection system, whereby queries are determined and transmitted to server 107 for transmission to user devices, and query selection data 112, data point value metrics 113, and response rate data 114 collect the relevant information, such as query selection (will this query be deployed to the caregiver?) data point value metrics (this shift is a better fit than that shift) and response rates (caregiver C does not respond to blood pressure queries when dealing with patient P.) Further training is provided in AI training system 110, primarily directed to query selection. In one instance, the response time for queries, quality of response, best time to send queries, etc. may be trained at AI training system 110, while general AI training may be provided at AI training system 105, also known as the data value algorithm. AI training system 105 may train on information such as shift availability, information needed from various patients, patient preferences, patient quantified problems (e.g. caregiver Y does not spend adequate time addressing the knee issue of client W, as reported by client W) and so forth. In this manner, targeted queries can be determined and provided to caregivers and patients.

The resulting system captures the personalized record of actionable data for each care recipient's home care history while maximizing the value, frequency, and accuracy of the data captured. The resultant system optimizes care using queries and feedback using a multiple variables and the processing described herein.

Thus according to one aspect of the present design, there is provided a caregiving system. The system includes a user device, a server arrangement connected to the user device, a query recording system connected to the server arrangement configured to record caregiver queries and responses to caregiver queries and provide recorded results to a storage element, and intelligence preparation hardware configured to collect data from the storage element and form caregiver queries based on query selection data, data point value metrics, and response rate information using at least one training system. Queries formed by the intelligence preparation hardware are provided to the server arrangement and user device to solicit query responses.

According to a further aspect of the present design, there is provided a patient caregiving system comprising a server arrangement configured to receive information and data from at least one user device, a data storage element comprising a query recording system configured to receive a care query from the server arrangement and store the care query, and a query processing module configured to receive query data from the data storage element and provide selected queries to the server arrangement, wherein the query processing module comprises a query selection system configured to employ query selection data, datapoint value metrics, and response rate data to select a query and provide the query to the server arrangement for transmission to a receiving user device.

According to another aspect of the present design, there is provided a method for providing caregiving information used in caring for a patient, comprising receiving information regarding the patient, recording the information received, and based on the information received, determining a query applicable to the patient, wherein the query comprises actionable data with respect to the patient. Determining the query applicable to the patient comprises employing artificial intelligence to determine possible care actions based on attributes of the patient and available care solutions, employing artificial intelligence training based on prior received applicable data to improve potential query related outcomes, and establishing the query based on query selection data, datapoint value metrics, and response rate data.

The above description is for the best presently contemplated modes of carrying out the invention. This description is not to be taken in a limiting sense, but is made merely for the purpose of illustrating general principles of embodiments of the invention. The scope of the invention is best defined by the appended claims. In certain instances, detailed descriptions of well-known devices, mechanisms and methods are omitted so as to not obscure the description of the present invention with unnecessary detail.

What is claimed is:

1. A system comprising:
a user device;
a server arrangement connected to the user device;
a query recording system connected to the server arrangement configured to record caregiver queries and responses to caregiver queries and provide recorded results to a storage element; and
intelligence preparation hardware configured to collect data from the storage element and form caregiver queries based on query selection data, data point value metrics, and response rate information using at least one training system;
wherein queries formed by the intelligence preparation hardware are provided to the server arrangement and user device to solicit query responses.

2. The system of claim 1, further comprising a notification system configured to prepare and transmit messages to a caregiver to complete a query when the caregiver does not respond to the query within an assigned time period.

3. The system of claim 1, wherein the system adds new data to a client record and places the new data in an index position based on a timestamp attached to the new data.

4. The system of claim 3, wherein the recorded results comprise a time series record of all raw physical, mental, social, and behavioral data generated by a patient's home care up to a current date.

5. The system of claim 1, wherein the intelligence preparation hardware comprises an artificial intelligence training system and a query selection system configured to select queries based on query selection data, datapoint value metrics, and response rate data.

6. The system of claim 1, wherein the intelligence preparation hardware comprises sparse vector and dense vector processing techniques.

7. The system of claim 1, wherein the intelligence preparation hardware comprises a neural network trained on client home care and query data with a goal of maximizing total data value when selecting query categories and query formats.

8. A patient caregiving system comprising:
a server arrangement configured to receive information and data from at least one user device;
a data storage element comprising a query recording system configured to receive a care query from the server arrangement and store the care query; and
a query processing module configured to receive query data from the data storage element and provide selected queries to the server arrangement, wherein the query processing module comprises a query selection system configured to employ query selection data, datapoint value metrics, and response rate data to select a query and provide the query to the server arrangement for transmission to a receiving user device.

9. The patient caregiving system of claim 8, further comprising an artificial intelligence data preparation component configured to receive data from the data storage element, process the data, and provide processed data to an artificial intelligence database.

10. The patient caregiving system of claim 9, further comprising a first artificial intelligence training system configured to train query intelligence based on information received from the artificial intelligence component hardware and provide query selection data, datapoint value metrics, and response rate date.

11. The patient caregiving system of claim 10, further comprising a second artificial intelligence training system configured to interface with the query selection system.

12. The patient caregiving system of claim 8, further comprising a notification system configured to prepare and transmit messages to a caregiver to complete a query when the caregiver does not respond to the query within an assigned time period.

13. The patient caregiving system of claim 8, wherein the patient caregiving system adds new data to a client record and places the new data in an index position based on a timestamp attached to the new data.

14. The patient caregiving system of claim 13, wherein recorded results comprise a time series record of all raw physical, mental, social, and behavioral data generated by a patient's home care up to a current date.

15. The patient caregiving system of claim 8, wherein the query processing module performs sparse vector and dense vector processing techniques.

16. The patient caregiving system of claim 10, wherein the first artificial intelligence training system employs a neural network trained on client home care and query data with a goal of maximizing total data value when selecting query categories and query formats.

17. A method for providing caregiving information used in caring for a patient, comprising:
receiving information regarding the patient;
recording the information received; and
based on the information received, determining a query applicable to the patient, wherein the query comprises actionable data with respect to the patient;
wherein determining the query applicable to the patient comprises:
employing artificial intelligence to determine possible care actions based on attributes of the patient and available care solutions;
employing artificial intelligence training based on prior received applicable data to improve potential query related outcomes; and
establishing the query based on query selection data, datapoint value metrics, and response rate data.
